(19) United States
(12) Patent Application Publication
(10) Pub. No.: US 2017/0281714 A1
(43) Pub. Date: Oct. 5, 2017

(54) METHOD OF TREATMENT USING LIPOSOMALLY FORMULATED REDUCED GLUTATHIONE TO COOPERATE WITH IL-10 TO MODULATE INFLAMMATORY RESPONSE TRIGGERED IN HIV+ AND TB IMMUNE-COMPROMISED DIABETIC PATIENTS

(71) Applicants: Your Energy Systems, LLC, Palo Alto, CA (US); Western University of Health Sciences, Pomona, CA (US)

(72) Inventors: Frederick Timothy Guilford, Palo Alto, CA (US); Vishwanath Venketaraman, Pomona, CA (US)

(73) Assignees: Your Energy Systems, LLC, Palo Alto, CA (US); Western University of Health Sciences, Pomona, CA (US)

(21) Appl. No.: 15/511,516

(22) PCT Filed: Sep. 28, 2015
(86) PCT No.: PCT/US2015/052719

§ 371 Date: Mar. 15, 2017

Related U.S. Application Data
(60) Provisional application No. 62/056,900, filed on Sep. 29, 2014.

(51) Int. Cl. A61K 38/06; A61K 9/127

(52) U.S. Cl. CPC A61K 38/063; A61K 9/127

(57) ABSTRACT

In the setting of chronic infection with a pathogen, the immune system must adapt to the ongoing presence of inflammatory responses driven by the pathogen in order to control the replication of the infecting agent at an acceptable level while limiting immune-mediated damage (immunopathology) (1). A complex interaction is required to accomplish this using a balance of immune related cytokines to stimulate and control the immune response. A particular formulation of reduced glutathione of high concentration in a liposome is proposed. In cooperation with the immune system, the liposomally formulated reduced glutathione according to the invention surprisingly achieves the balance between over-reduction of IL-10 and its necessary bodily function and signaling.

### CONTINUATION DATA

For U.S. purposes, this application claims benefit of, and as required, is a continuation-in-part of U.S. Provisional Application 60/596,171 filed on Sep. 6, 2005 entitled Enhanced Method And Composition For The Treatment Of HIV+ Tuberculosis Patients With Anti-Retroviral Drugs And Liposomal Encapsulation For Delivery Of Reduced Glutathione and of U.S. Provisional Application 60/824,671 filed on Sep. 6, 2006 Enhanced Method And Composition For The Treatment Of HIV+ Tuberculosis Patients With Anti-Retroviral Drugs And Liposomal Encapsulation For Delivery Of Reduced Glutathione , and is intended to be a continuation-in-part or the substantive equivalent in any regional or national stage in which continuation is permitted to preserve an earlier filing date, and is a continuation in part of U.S. application Ser. No. 13/458,449 which is a continuation in part of Ser. No. 12/065,753, which was a continuation in part of then pending application Ser. No. 11/163,979 filed Nov. 6, 2005 claiming priority from 60/522,785 filed Nov. 7, 2004, which applications are adopted by reference

### TECHNICAL FIELD

The technical field involves diabetes and treatments to cooperate with body cytokines to modulate immune response and inflammatory effects caused by diabetes and describes method of utilizing a particular formulation of high concentration reduced glutathione in a liposome of enhanced intra-liposomal concentration to achieve a surprising effect of cooperation with a cytokine IL-10 to modulate, but not over-regulate, inflammatory response in immune-compromised diabetic patients, especially those who are HIV+ or have tuberculosis while simultaneously preserving necessary IL-10 response.

### SUMMARY OF INVENTION

### Technical Problem

Elevated IL-10 levels have been observed to occur in a number of chronic infectious diseases in humans, including visceral leishmaniasis, leprosy and tuberculosis (1). Increased levels of IL-10 have been shown to occur in HIV disease and these elevated IL-10 levels correlated with a more advanced disease stage and progression of the illness (1). Recent data shows that IL-10 is also elevated in Type 2 Diabetes Mellitus (T2DM). Monocytes are a major source of IL-10 in HIV-infected individuals and they produce this cytokine in significantly higher amounts than in HIV-negative controls (1). IL-10 has been shown to be an immunosuppressive cytokine and may hinder an individual's ability to resolve an infection by down-regulating protective immune responses. Thus, facially, it would make sense to decrease IL-10 levels.

A typical pharmacological model to reduce the effect of a body chemical is to block a receptor for that chemical or to chemically combine with a particular chemical to inhibit its usefulness, or to directly change the chemical. In the instance of IL-10, this would be dangerous because IL-10 can have positive effects.

However, IL-10 can also stimulate the proliferation of B cells and enhance their maturation into plasma cells, and may enhance the cytotoxicity of CD8 T cells. Thus, IL-10 can have complex, pleiotropic effects on the immune system, involving multiple cell types and immune compartments (reviewed in (1).

At the same time, some function of IL-10 is needed to avoid infections such as severe immunopathology in Toxoplasma gondii and malaria models (1) Thus, facially, it would make sense to increase IL-10 levels.

So, it appears that an excess elevation of IL-10 can lead to compounding of infection or undesirable inflammatory response, while some level of IL-10 is needed for protection. There is no medical resolution or explanation of this dichotomy.

The technical problem is to utilize a composition to enable the body to find an intermediate ground whereby the body can be induced to properly and continuously regulate its immune system to the proper levels in the face of the substantial challenges of diabetes and other systemic challenges found in daily life, particularly in HIV+ and Tuberculosis patients which need balanced and enhance IL-10 responses.

### Solution to Problem

The inventors propose that liposomally formulated reduced glutathione in significant concentration in the liposome be utilized to cooperate with a measured immune response indicated by IL-10 levels in order to provide a nuanced level of IL-10 response that suppresses an over-reaction of inflammatory effect, but allows a “gateway” for necessary levels of inflammatory response protection that a properly regulated IL-10 level encourages. The invention is particularly useful in diabetic patients and especially in such patients faced with HIV or mycobacteria challenges such as tuberculosis or other bacterial or viral infection.

The invention enables oral treatment with a composition that restores glutathione function to cooperate with IL-10 to achieve a proper balance with minimal or no side effects compared with known treatments and compositions that have been effective. At present there is no known treatment for this problem of unbalanced IL-10 that has minimal or no side effects. Further, the method of treatment and the use of the composition for treatment can occur in conjunction with other traditional treatments as an adjuvant.

### ADVANTAGEOUS EFFECTS OF INVENTION

In the setting of chronic infection with a pathogen, the immune system must adapt to the ongoing presence of inflammatory responses triggered by the pathogen in order to control the replication of the infecting agent at an acceptable level while limiting immune-mediated damage (immunopathology) (1). A complex interaction is required to accomplish this using a balance of immune related cytokines to stimulate and control the immune response. A particular formulation of reduced glutathione of high concentration in a liposome is proposed. In cooperation with the immune system, the liposomally formulated reduced glutathione according to the invention surprisingly achieves the balance between over-reduction of IL-10 and its necessary bodily function and signaling.

### BRIEF DESCRIPTION OF DRAWINGS

FIG. 1 is a bar graph showing the increased elevation of IL-10 in the plasma of individuals with Type 2 Diabetes Mellitus versus health individuals.

FIG. 2 is a bar graph showing the level of IL-10 after administering liposomal glutathione in HIV patients over 13 weeks.

FIG. 3 is two bar graphs using various substances or compositions for raising glutathione in macrophages and how macrophages respond to these various methods. On the left is a control graph for healthy individuals. On the right is a graph for individuals affected with Type 2 diabetes mellitus.

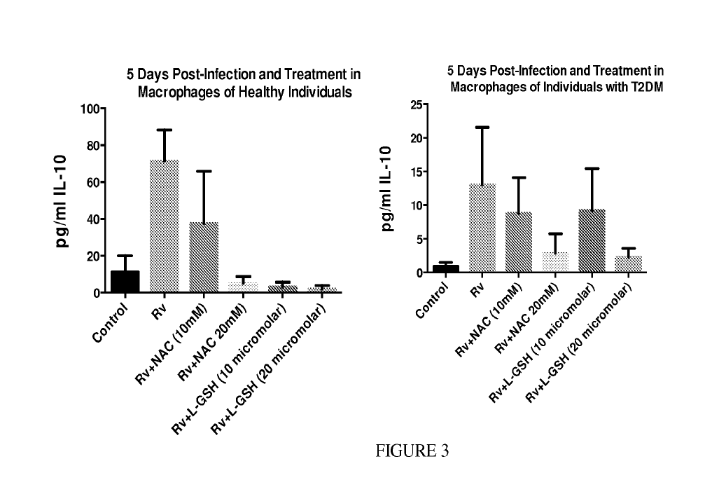

The left graph of FIG. 3 shows that L-GSH (liposomal glutathione) is 1,000 times more effective in decreasing IL-10 than NAC, a precurosor of glutathione.

The right graph of FIG. 3 depicts the response of macrophages from individuals with Type 2 diabetes mellitus ex vivo, which have been infected with M. Tb strain H37Rv. The x axis shows the affect of various methods of raising glutathione in these cells.

### FURTHER DETAILED DESCRIPTION OF EMBODIMENTS

Formulation of Composition and Dosages for Method

The method of treatment, or alternatively, the use of the composition described in this application for treatment, is for patient populations that are diabetic, particularly Type-2 Diabetes Mellitus. More particularly, the patient populations most benefitted are diabetic patients with diabetes who are human immunodeficiency virus positive (HIV+). Diabetic patient populations who have M. tb (tuberculosis) are benefitted. Diabetic patient populations who are both HIV+ and have tuberculosis are benefitted. HIV+ patients are benefitted. Oral liposomally encapsulated reduced glutathione for these patient populations is uniquely designed to be absorbed a) across the mucosa of the nose, mouth, gastrointestinal tract, b) after topical application for transdermal, or c) by intravenous infusion of glutathione with or without liposome encapsulation is prepared under the method and according to the composition described as follows:

Basic Dosing Information

For a typical adult ranging from 55 kg to 90 kg, the dose of oral liposomally encapsulated reduced glutathione is oral liposomally encapsulated reduced glutathione 422 mg (1 teaspoon) (5 ml each) of concentration of approximately 8.25% w/w or 84 mg/ml at least twice a day. It could be any concentration above 3.3% w/w within the liposomes normally in increments of 0.5% w/w between 3.3% w/w and 9% w/w or higher. There are approximately 423 mg. of reduced glutathione per teaspoon but maybe 420 or 428 mg per teaspoon. Administration may be oral, by inhalation, mucosal, rectal, or intravenous administration.

It has been shown that 1.5 teaspoons twice a day will decrease IL-10 to normal levels in individuals with uncomplicated (meaning their condition is stable and not life threatening) individuals. In individuals with an acute, severe infection, it may be preferable to administer 4(four) teaspoons (5 ml each) 4 times per day.

### BACKGROUND INFORMATION AND ART RELATED TO THE INVENTION

In the setting of chronic infection with a pathogen, the immune system must adapt to the ongoing presence of inflammatory responses triggered by the pathogen in order to control of the replication of the infecting agent at an acceptable level, while limiting immune-mediated damage (immune related pathology) (1). A complex interaction is required to accomplish this using a balance of immune related cytokines to stimulate and at the same time, control the immune response. A particular formulation of reduced glutathione of high concentration in a liposome is proposed. In cooperation with the immune system, the liposomally formulated reduced glutathione according to the invention surprisingly achieves the balance between over-reduction of IL-10 and its necessary bodily function and signaling.

Cytokines are small proteins that are released by cells and affect the behavior of other cells and sometimes may affect the releasing cell itself. They are important in health and disease, specifically in host responses to infection. The term “interleukin” was initially used by researchers for those cytokines whose presumed targets are principally leukocytes which are white blood cells. Cytokines have an important role in the adaptive immune response as both effectors and regulators of immunity, with the immune response to the bacteria Mycobacterium tuberculosis offered as an example in this discussion. The expression profile in CD4+ T cells clearly delineates the dominant Th1 -like response that is associated with control of infection. When produced in appropriate quantities, proinflammatory cytokines (described below) play a beneficial role as mediators of host resistance to infectious agents (3).

Cytokines needed for infection control include those associated with both the innate and adaptive immune system. The innate immune system is comprised of cells such as macrophage, antigen presenting and neutrophil, which make up the first line of defense against invading organisms and responds in a non-specific manner. The cytokines associated with the innate immune response include TNF-α, IL-1, IL-10, IL-12, type I interferons (IFN-α and IFN-β), IFN-γ, and chemokines. A more specific response to the invader is defined in the adaptive immune response which has specific antibodies generated to attach to the invader and direct immune defense.

Macrophages (Greek: big eaters,), are a type of white blood cell that engulf and digest cellular debris, foreign substances, microbes, and cancer cells in a process called phagocytosis. They play a critical role in both non-specific defense (innate immunity) and also help initiate specific defense mechanisms (adaptive immunity). Neutrophils are a type of white blood cell also known as Neutrophil granulocyte cell (also known as neutrophils) are the most abundant (40% to 75%) type of white blood cells in mammals and form an essential part of the innate immune system and serve to respond in the beginning (acute) phase of inflammation, particularly as a result of bacterial infection. Neutrophils are one of the first-responders of inflammatory cells to migrate towards the site of inflammation. The cytokine Tumor Necrosis Factor alpha (TNF-α) is produced by activated macrophages in response to microbes. TNF-α mediates the recruitment of neutrophils and macrophages to sites of infection by stimulating endothelial cells to produce adhesion molecules and by producing chemokines which are chemotactic cytokines. TNF- a also acts on the hypothalamus to produce fever and it promotes the production of acute phase proteins (4).

Interleukin 1 (IL-1) is another inflammatory cytokine produced by activated macrophages. Its effects are similar to that of TNF-α and it also helps to activate T cells (4).

Interleukin 12 (IL-12) is produced by activated macrophages and dendritic cells. It stimulates the production of IFN-γ and induces the differentiation of T helper (Th) cells to become Th1 cells. IFN-γ functions to initiate the move to adaptive immunity. In addition, it enhances the cytolytic (killing by bursting or impairing the membrane of a cell) functions of cytotoxic (Tc) and natural killer (NK) cells (4).

Interleukin 10 (IL-10) is produced by activated macrophages and Th2 cells. An activated macrophage is simply defined as cells that secret inflammatory mediators and functions to kill intracellular pathogens. Exposure of macrophages to classical activating signals like bacterial products such as lipopolysaccharide (LPS) in the presence of immunoglobulin G (IgG) immune complexes induces the production of a macrophage cell type that is fundamentally different from the classically activated macrophage. These cells generate large amounts of IL-10 and as a result, were potent inhibitors of acute inflammatory responses to bacterial endotoxin (5).

Th-1 cells can be contrasted with Th-2 cells by describing Th1 cells as cells which produce interferon (IFN)-γ, interleukin(IL)-2 and (TNF)-β, and evoke cell-mediated immunity and phagocyte-dependent inflammation. Th-2 cells as cells, which produce IL-4, IL-5, IL-6, IL-9, IL-10, and IL-13, evoke strong antibody responses (including those of the IgE class) and eosinophil accumulation, but inhibit several functions of phagocytic cells which creates a phagocyte-independent form of inflammation(6). IL-10 is predominantly an inhibitory cytokine. It inhibits production of IFN-γ by Th1 cells, which shifts immune responses toward a Th2 type(4). Too much Th2 domination of response may result in damage to normal tissues and shows up in conditions such as progressive systemic sclerosis and lung fibrosis. Excessive Th2 response can even favor a more rapid evolution of HIV infection toward the full blown manifestation of the disease. IL-10 also inhibits cytokine production by activated macrophages and the expression of class II major histocompatibility complex (MHC) and co-stimulatory molecules on macrophages, resulting in a dampening of immune responses. As IL-10 is primarily an anti-inflammatory cytokine, it has critical functions in preventing inflammatory and autoimmune diseases; it has emerged as a key immunoregulator during infection with viruses, bacteria, fungi, protozoa, and helminthes (1) Inflammatory bowel disease and other excessive inflammatory responses occurring in IL-10^{-/-} (knockout)mice indicate that IL-10 is critically involved in limiting deleterious inflammatory responses in vivo (1). However, complete knock out of IL-10 can result in severe infection from a variety of sources Inflammatory responses that progress to the point of damaging normal tissues are often referred to as chronic inflammation. While IL-10 formation is needed to limit damage from chronic inflammation, too much IL-10 can cause a loss of immune control and progression of infection. Thus, IL-10 can have complex, pleiotropic effects on the immune system, involving multiple cell types and immune compartments (1). Studies have been performed to evaluate the advantage of elevating the level of IL-10 in conditions associated with increased inflammation such as rheumatoid arthritis, inflammatory bowel disease, psoriasis and chronic Hepatitis C (6). This paper is extensive in its review of IL-10. The effect of increasing IL-10 as an anti-inflammatory using recombinant IL-10 was varied among patients and no consistent pattern emerged. (6). This extensive review paper on IL-10 does not mention a method of modulating or decreasing the level of IL-10 (6).

The following is an example of the need for a cytokine to enable cells to function normally, but at the same time an excess of the cytokine may be detrimental if produced in larger than needed quantities. A similar need for a cytokine to facilitate intracellular killing of bacterial has been shown in regard to the need for TNF-α , which is needed to facilitate intracellular killing of Mycobacterium tuberculosis. At the same time, too much TNF-α is associated with chronic inflammation damage to tissues and is now the target of monoclonal antibodies designed to bind and decrease the amount of TNF-α in individuals with chronic arthritis. Interestingly, L-GSH treatment of H37Rv-infected neutrophils did not alter the synthesis of IL-6 and TNF-α needed for intracellular killing of bacteria; L-GSH treatment prevents an excess of TNF-α that can leak out of the cell and stimulate damage in other normal tissues which may be detrimental. It has been shown that the use of a monoclonal antibody specific to TNF-α , which will significantly reduce the availability of TNF-α for control of arthritis symptoms can become a risk factor for infection with Mycobacterium tuberculosis M. Tb. (7). IL-10 is capable of inhibiting synthesis of pro-inflammatory cytokines such as IFN-γ, IL-2, IL-3, TNFα and Granulocyte macrophage colony-stimulating factor (GM-CSF) made by cells such as macrophages and regulatory T-cells. IL-10 deactivates macrophages directly by influencing macrophage recruitment, viability, morphology, phagocytosis, expression of cytokine receptors and major histocompatibility complex molecules, antigen presentation, production of monokines, generation of reactive oxygen and nitrogen intermediates, and killing of microbes and tumor cells (3). This activity can inhibit peripheral blood monocuclear cells (PBMC) responses to a variety of organisms including fungal antigens such as Candida albicans (3). The defense against invasion by organisms can include the release of TNF-α and IL-1β from PBMC. It has been shown that IL-10 can inhibit the release of these proinflammatory cytokines and allow the continuation of growth of the invading organism. The ability of IL-10 to inhibit cytokine production by T cells and NK cells is thought to be largely indirect, by alteration of monocyte/macrophage functions(1).

Elevated IL-10 levels have been observed to occur in a number of chronic infectious diseases in humans, including visceral leishmaniasis, leprosy and tuberculosis (1). Increased levels of IL-10 have been shown to occur in HIV disease and these elevated IL-10 levels correlated with a more advanced disease stage and progression (1). Monocytes are a major source of IL-10 in HIV-infected individuals and this cytokine is produced in significantly higher amounts than in HIV-negative controls (1).

IL-10 has been shown to inhibit release of fungus-stimulated TNF-α at the transcriptional level, which may contribute to its ability to inhibit macrophage microbicidal activity against a variety of fungal pathogens (3).

IL-10 has also been shown to be upregulated in various types of cancer. The biological role of IL-10 in cancer is quite complex; however, the presence of IL-10 in advanced metastases and the positive correlation between serum IL-10 levels and progression of disease indicates a critical role of IL-10 in the tumor microenvironment. In this situation the excess of IL-10 may induce an immune suppression or induce a tolerance to the tumor tissue. The ability of L-GSH, the present invention to diminish IL-10 may be useful in reducing the immune suppression that occurs in response to cancer and at the same time help arm macrophages to function normally in the phagocytosis and killing of tumor cells.

So, it appears that an excess elevation of IL-10 can lead to infection, while some level of IL-10 is needed for protection.

Recent unpublished studies in the laboratory of Vishwanath Venketaraman, PhD at Western University have confirmed the elevation of IL-10 in the plasma of individuals with HIV. The studies went on to show that the addition of liposomal glutathione (produced by Your Energy Systems, LLC) was able to lower the level of IL-10 back to levels associated with non-infected individuals.

IL-10 has previously been shown to be elevated in type 2 diabetes mellitus. Prior to the current invention, no method of lowering IL-10 in individuals with type 1 or type 2 diabetes mellitus has been proposed. Type 1 diabetes is an immune disorder in which the body's immune cells (white blood cells) attack and destroy insulin-producing beta cells in the pancreas. As a result, the body cannot produce insulin and glucose stays and builds up in the blood, where it damages all the organ systems. Type 2 diabetes is a disorder in which either the body does not produce enough insulin, or the cells ignore the insulin. Similar to type 1 diabetes, type 2 causes a build-up of glucose in the blood which damages the body's organ systems. One of the key complications of Type 2 diabetes is prolonged infection and inhibited response to infection and poor wound healing. This Type 2 form of the disease is commonly referred to as adult-onset diabetes. Cytokine levels, such as for IL-10, in plasma samples can be determined by sandwich enzyme linked immunosorbent assay (ELISA) using assay kits from eBioscience (kit named ELISA Ready-Set-Go).

Individuals with type 2 diabetes mellitus are susceptible to increased incidence of complications and infections than the non-diabetic population. The causes of the increased infection may be related to changes in the immune cell defense against infection. While there are numerous causes of infection in individuals with Type 2 diabetes mellitus, the suppression of the inflammatory cytokines due to elevation of IL-10 contributes to the persistence and progression of infection.

The finding of elevation of serum levels of IL-10 in individuals with Type 2 Diabetes mellitus suggests that elevation of IL-10 may be associated with the increased risk of infectious complications seen in this group. IL-10 goes even higher during infection as shown in cell culture introduction of M.tb bacteria (tuberculosis) to the macrophages from individuals with type 2 diabetes mellitus in the lab of V. Venketaraman, PhD. A method of lowering IL-10 during infection and at the same time supporting an efficient, non-damaging immune function may offer a significant advantage for prevention and management of complications such as infection in individuals with Type 2 Diabetes mellitus.

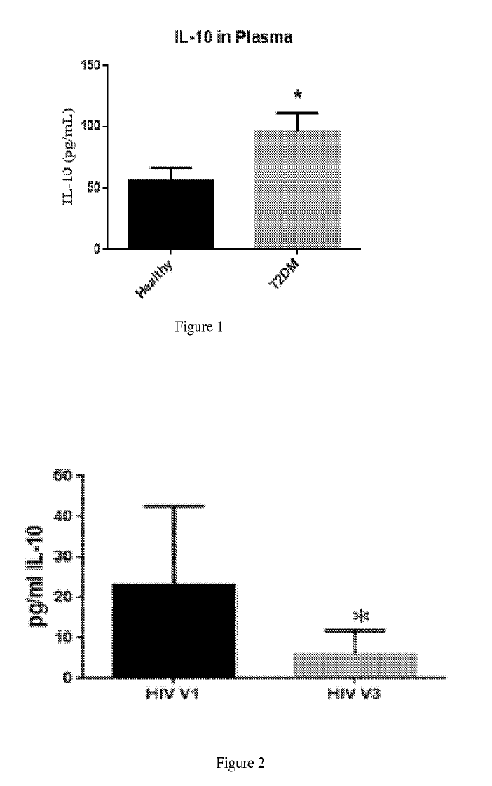

The present invention, the oral administration of liposomal glutathione(LGSH) from Your Energy Systems, LLC (YES) of Palo Alto, Calif., and available for purchase from that company, has been shown to cooperate with IL-10 to return elevated serum levels of IL-10 to more normal levels. This is particularly true after intracellular infection, where it has been shown that IL-10 increases during infection in cells from both normal (left) and Type 2 Diabetes mellitus (right) as shown in FIGS. 1, 2 and 3.

In FIG. 3, the left graph depicts the response of macrophages from healthy individuals ex vivo, which have been infected with M. Tb strain H37Rv. The x axis shows the effect of various methods of raising glutathione in these cells. The graph on the right shows a similar effect of infecting macrophage cells ex vivo from individuals with T2DM with the effect of various methods of raising glutahtione. The x axis denots various material applied to these cells and the resuling reponse of IL-10 in μg/ml is shown. The graph shows that L-GSH (liposomal glutathione) is 1,000 times more effective in decreasing IL-10 than NAC, a precurosor of glutathione. Abbreviations: NAC—N-acetly cysteine, L-GSH—liposomal glutathione, RV-Mycotplasma tuberculosis H37Rv, μg/ml—picogram per milliliter., mM—milliMolar, —microM—microMolar.

The oral administration of orally ingesting 2 teaspoons (840 mg GSH in liposomes) of LGSH from YES to individuals with HIV+ disease has previously been shown to restore depleted glutathione levels in lymphocytes back to normal levels using lymphocytes from HIV+ individuals, whose cells are depleted of glutathione (Venketaraman, Western Univ 2014, unpublished). The dose used in the current invention is 2 teaspoons (840 mg GSH in liposomes) twice a day taken orally.

As shown in FIG. 3, raising glutathione levels with NAC can also reduce to normal the levels of IL-10 from macrophages in cell culture. The data also shows that LGSH is 1,000 times more efficient in lowering the levels of IL-10 secreted from macrophages compared to NAC. NAC supplied in millimolar amounts lowered the cell culture IL-10 secretion level, while LGSH in micromolar (1,000 times less) resulted in similar if not lower secretion levels.

A study by Morris and Venketaraman et al at Western University investigated the effect of N-acetyl cysteine (NAC) and liposomally encapsulated glutathione to prevent the replication of intracellular Mycobacterium tuberculosis after infecting the cells with the organism (2). Previous work by Venketaraman has shown that raising glutathione levels with NAC in this cell culture model will limit the growth of Mycobacterium tuberculosis (TB). The study shows that both NAC and liposomally encapsulated glutathione were able to limit the growth of the organisms to a level below 1000 colony forming units per milliliter (CFU/ml). NAC at 10 millimolar reduced the CFU/ml to 8,000, while the liposomally encapsulated glutathione at 5 micromolar concentration reduced the CFU/ml to 6,000 CFU/ml. This data demonstrates that liposomally encapsulated glutathione is over 2000 times more potent than NAC in maintaining the function of macrophages undergoing the oxidative stress of an intracellular infection.

The study shows that liposomally encapsulated reduced glutathione formulated per this invention has a significantly increased absorption and function in the macrophages from individuals with HIV that are undergoing infection with M. tb (Mycobacterium tuberculosis). The absorption of the liposomally encapsulated glutathione is 1000× (one thousand times) more efficient than the glutathione precursor N-acetyl cysteine (NAC) in restoring normal glutathione levels and restoring the glutathione related function of slowing the replication of M tb in macrophages taken from individuals with HIV. “Glutathione Supplementation Improves Immune Function in HIV+ Macrophages,” Morris D, Guerra C, Khurasany M, Guilford T, Venketaraman V, J Interferon Cytokine Res. 2013 May;33(5):270-9. PMID Epub 2013 Feb. 14 (Western University of Health Sciences, Pomona, Calif. 91766, USA) PMID 23409922(“Morris D”) (2).

The surprising and novel finding in the Morris D et al study of the dramatic absorption of liposomally encapsulated reduced glutathione compared to N-acetyl cysteine (“NAC”) explains the ability of this formulated form of liposomally encapsulated reduced glutathione to restore macrophage function, lower IL-10 levels back to normal and restore the function of macrophages to the “classically” described form known as M1 function.
“In a previous study we observed elevated levels of TGF-β in both the plasma and macrophage culture supernatants of HIV+ macrophages. This elevated TGF-β will compromise the amount of GCLC present inside the cell; consequently, supplementing the raw materials [such as with NAC] for de novo synthesis in HIV+ individuals who are over expressing TGF-β will not result in the same increased production of reduced GSH that is observed in individuals who are not over expressing TGF-β. In addition, this phenomenon may explain why lGSH [the liposomally encapsulated reduced glutathione of this invention] at lower concentrations than NAC is more effective at raising the concentration of reduced GSH in HIV+ macrophages than in HIV− macrophages. Supplementing with an lGSH formulation provides complete GSH molecules to cells, circumventing the enzymatic pathway responsible for GSH production, without the requirement for the cell to construct the tripeptide. This may also explain why treatment with lGSH seems to raise the ratio of reduced GSH to GSSG at much lower concentrations than NAC, as cells treated with NAC will have to produce new molecules of reduced GSH utilizing their own enzymatic machinery. [emphasis added, citation omitted].” Morris et al. (2)

The ability to restore IL-10 and maintain normal immune cell function by raising glutathione directly during an infectious process in the cell is novel and has not been previously reported. The observation that liposomally encapsulated glutathione is 2000 (two-thousand) times more effective in maintaining glutathione and the ability of the cell to limit replication of an intracellular infectious agent such as TB is also novel and previously unreported.

Other indications for which treatment is appropriate by liposomal reduced glutathione or for which administering liposomal reduced glutathione is beneficial include signs of infection that may be bacterial such as high fevers, increased white blood count on blood sample testing, findings consistent with pneumonia on physical examination, severe illness requiring administration to the hospital of intensive care unit for conditions that may be accompanied by infection, the finding of low reduced glutathione on blood testing or similar indication of oxidant stress.

The concentration of the glutathione in the liposomes can be in a range from 3.3% w/w to 9% w/w or higher. The concentration can be 3.3% w/w, 4% w/w, 5% w/w, 6% w/w, 7% w/w, 7.5% w/w, 8% w/w, 8.5% w/w or 9% w/w. The amount of 3.3% w/w is equivalent to a concentration of 123mM. 8.25% w/w is preferred.

Deionized water can be used to bring w/w percentages up to 100% w/w in any of the tables or formulations below.

DETERMINE INDIVIDUAL DOSE BY BODY WEIGHT: For children
Under 30 lbs: ¼-½ teaspoon=100-200 mg GSH
30-60 lbs: ½-1 teaspoon=210-420 mg GSH
60-90 lbs: ¾-1.5 teaspoon=316 mg-630 GSH
90-120 lbs: 1-2 teaspoon=422-844 mg GSH
120-150 lbs: 1½-3 teaspoon=630-1260 mg GSH
Over 150 lbs: 1½-3 teaspoons=630-1260 mg GSH
The invention should be used on a continuous basis.
Children - should use a dose of liposomally encapsulated reduced glutathione equivalent to 60 mg/Kg of body weight daily in divided doses.
These doses should be continued for the duration of the duration of the illness and for purposes of maintaining adequate glutathione in tissues before, during and after therapy for the infective agent.
The components of this invention can be administered separately or combined in a single capsule or dose.

### EXPERIMENTAL RESULTS SHOWN IN FIGURES

FIG. 1 is a bar graph showing the increased elevation of IL-10 in the plasma of individuals with Type 2 Diabetes Mellitus versus health individuals. The x-axis has bar graphs for healthy versus Type 1 diabetes patient populations. The y-axis has levels of IL-10 in μg/ml. The results show that the level of IL-10 is elevated in the plasma of individuals with T2DM compared to healthy individual.

FIG. 2 is a bar graph showing the level of IL-10 after administering glutathione in HIV patients over 13 weeks. The x-axis is for an initial population at the outset (time 0) setting a baseline and the second bar is for the same populations 13 weeks later after a third visit (V3). The y-axis depicts the level of IL-10 in μg/ml. During the 13 weeks, patients have taken 1.5 teaspoons of liposomal glutathione 2 times a day for 13 weeks. The results show a return of IL-10 to a lower, more normal level. The asterisk (*) is used to denote a statistically significant change p<0.02 in the values p<0.02.

FIG. 3: FIG. 3 is two bar graphs using various substances or compositions for raising glutathione in macrophages and how macrophages respond to these various methods. On the left is a control graph for healthy individuals. On the right is a graph for individuals affected with Type 2 diabetes mellitus.

The left graph depicts the response of macrophages to various methods of raising glutathione in cells on the x axis. from healthy individuals ex vivo, which have been infected with M. Tb strain H37Rv. The x axis shows the affect of various methods of raising glutathione in these cells. The left graph shows that L-GSH (liposomal glutathione) is 1,000 times more effective in decreasing IL-10 than NAC, a precurosor of glutathione.

The right graph depicts the response of macrophages to various methods of raising gluathione in cells on the x axis from individuals with Type 2 diabetes mellitus ex vivo, which have been infected with M. Tb strain H37Rv.

The y axis in the graphs shows the level of IL-10 in μg/ml.

Abbreviations: NAC—N-acetly cysteine, L-GSH—liposomal glutathione, RV—Mycoplasma tuberculosis H37Rv, μg/ml—picogram per milliliter., mM—milliMolar, -microM—microMolar.

### DETAILED DESCRIPTION OF THE PREFERRED EMBODIMENTS

The methods of manufacture described in Keller et al U.S. Pat. No. 5,891,465, U.S. Pat. No. 6,610,322, and U.S. Pat. No. 6,726,924 and U.S. Provisional application No. 60/597,041 by this inventor are adopted herein and into the modes of this invention and can be applied to the examples without undue experimentation. Liposomal formulations preferred in this invention can be purchased from Biozone, Inc. of Pittsburgh, Calif. Reduced glutathione can be purchased from Sigma-Aldrich of St. Louis, Mo. or from Kyowa Hakko USA, Inc., 767 3^{rd} Ave. No. 9, of New York City, N.Y. 10017 with a Western regional office at 85 Enterprise, Suite 430, Aliso Viejo, Calif. 92656. Liposomally encapsulated reduced glutathione can be purchased from Your Energy Systems, LLC, 555 Bryant St., Suite 305, Palo Alto, Calif. 94301.

### EXAMPLE 1

Liposomal glutathione Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

A lipid mixture having components lecithin, and glycerin were commingled in a large volume flask and set aside for compounding. Hydroxylated lecithin is the preferred ingredient. In a separate beaker, a water mixture having water, glycerin, glutathione were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al Pat #5,891,465, U.S. Pat. No. 6,958,160 and U.S. Pat. No. 7,150,883 and U.S. provisional application No. 60/597,041 are incorporated in this description. Concentrations of liposomally encapsulated glutathione from 3.3% w/w, 4% w/w, 5% w/w, 6% w/w, 7% w/w, 7.5% w/w, 8% w/w, 8.5% w/w or 9% w/w liposomally encapsulated glutathione may be formed and utilized for dosing by decreasing the amounts of glutathione and preplacing the material with an increase in the sterile water concentration.

### Example 1A

Liposomally encapsulated reduced glutathione Drink or Spray 2500 mg Per Ounce or Form Suitable for Encapsulation or Gel: In %, according to w/w: Deionized Water 75, Glycerin 15.00, Lecithin 1.50, Extract Potassium Sorbate 0.10, Glutathione 8.5 (reduced)

A lipid mixture having components lecithin, ethyl alcohol and glycerin were commingled in a large volume flask and set aside for compounding. Hydroxylated lecithin is the preferred ingredient.

In a separate beaker, a water mixture having water, glycerin, glutathione were mixed and heated, but not to more than 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. A spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description. Concentrations of liposomally encapsulated glutathione from 3.3% w/w, 4% w/w, 5% w/w, 6% w/w, 7% w/w, 7.5% w/w, 8% w/w, 8.25%, 8.5% w/w or 9% w/w liposomally encapsulated glutathione may be formed and utilized for dosing by decreasing the amounts of glutathione and preplacing the material with an increase in the sterile water concentration.

### EXAMPLE 2

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing biologically encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% w/w on the interior. The concentration of water in a liposome formulation can range from 60-90% water. An essential component of the present invention is the formulation of a liposome with a relatively small amount of water, in the range of 5-10% w/w. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% w/w range.

The methods described by Keller et al, U.S. Pat. No. 6,726,924 are incorporated in this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Filling of gel caps on a large scale is best with the rotary die method or others such as the Norton capsule machine.

### EXAMPLE 3

Embodiment number four of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description. Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self-assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ. references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

The low molecular weight in the preferred embodiments more effectively deliver the liposomally encapsulated reduced glutathione in active reduced form as needed and thus result in the surprising effect of the invention. The absorption into cells is a particular advantage of the preferred embodiment of the invention.

Another refinement for producing the composition for the method or use of this invention is reduced glutathione in a self-forming liposome sold under the brand name “QuSome” ® by Biozone Laboratories, Inc. of Pittsburgh, Calif. The Qusome self-forming liposome can be formed containing reduced liposomally encapsulated glutathione in a concentration of 5% reduced glutathione encapsulated in the liposome. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. Pat. No. 6,958,160 and U.S. Pat. No. 7,150,883 are incorporated in this description. The most important details of that manufacturing are as follows: The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise, a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops.

The compositions may be administered topically, inter-orally, vaginally or rectally.

PEG-12 Glyceryl Dioleate was obtained from Global 7 (New Jersey) for the following formulations. This can be substituted for the lecithin w/w% as needed to accomplish the formulation, or applied as set forth below.

In the following formulations, the “set percentage” w/w% of reduced glutathione is selected from 3.3%, 4%, 5%, 6%, 7%, 7.5%, 8%, 8.5% or 9% or amounts approximately to those percentages.

### EXAMPLE 5A

Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal encapsulation formulation, the preferable mode is to add flavor or other taste masking ingredients after liposomal encapsulation formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

### EXAMPLE 5B

Spontaneous Liposomes for Intravenously Administered Therapeutic Compound and as a Drug Solubilization Vehicle for use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w. Deionized water is added and gently mixed. Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered.

The concentration of liposomally encapsulated glutathione in the liposomes resulting from the Qusome formulation is 5% w/w for topical application. It is possible to use the Qusome technology in creating an oral formulation also and the 8.25% glutathione in w/w concentration encapsulated in the liposome may be used in the oral formulation.

### FURTHER EXAMPLES

### EXAMPLE 6

Treatment of infants with liposomal glutathione The recommended dose of liposomal glutathione for infants is 3 mg/l pound (2.2 Kg) to be taken orally or by nasogastric tube in the infant formula or water. A similar dose may be administered intravenously for cases where oral feeding or nasogastric tube feeding is not possible. A patient example is that a neonate suffering from wheezing was given approximately 6 drops per feeding with 4 feedings per day of concentration of approximately 123 mM liposomal glutathione. 20 Drops is considered one cc.

For a typical adult with diabetes ranging from 55 kg to 90 kg, the dose of oral liposomally encapsulated reduced glutathione is oral liposomally encapsulated reduced glutathione 422 mg (1 teaspoon) (5 ml each) of concentration of approximately 8.25% w/w or 84 mg/ml at least twice a day.

For diabetes patients with Tuberculosis or other infection with a bacteria or a virus

For a typical diabetic adult ranging from 55 kg to 90 kg, the dose of oral liposomally encapsulated reduced glutathione is oral liposomally encapsulated reduced glutathione 422 mg (1 teaspoon) (5 ml each) of concentration of approximately 8.25% w/w or 84 mg/ml at least twice a day. More preferable is administration of 4 teaspoons (5 ml each) 4 times per day. If tolerated well, a loading dose of another teaspoon (5m1) after perhaps an hour would be helpful.

For diabetes patients with HIV

For a typical adult with ranging from 55 kg to 90 kg, the dose of oral liposomally encapsulated reduced glutathione is oral liposomally encapsulated reduced glutathione 422 mg (1 teaspoon) (5 ml each) of concentration of approximately 8.25% w/w or 84 mg/ml at least twice a day. A dose of one and one-half (1.5) teaspoons twice a day has been shown to raise glutathione in individuals with HIV. The effect of this dose in a human taking this dose over a thirteen week period is depicted in FIG. 2.

For diabetes patients with HIV and Tuberculosis or HIV+ only

The recommended dosage is the same as for diabetic patients with tuberculosis. Dosages can be increased up to 4 times 4 teaspoons (5 ml each) per day for these particularly immunologically challenged patients depending on patient tolerance.

### INDUSTRIAL APPLICABILITY

The industrial applicability is to treat individuals with elevations of IL-10, in particular as a result of diabetes and diabetes in combination with other diseases.. The surprising effects are most particularly seen in difficult patient populations such as individuals with diabetes, or diabetes and tuberculosis or in individuals with HIV, or with diabetes and HIV, or with diabetes, HIV and tuberculosis, all of whom are at substantial risk for infection and need IL-lO levels controlled in a nuanced way to promote proper, but not excessive inflammatory responses, such as to infection, for which they are at increased risk.

### Summary

The use of oral administration of LRG for lowering IL-10 levels in individuals who are undergoing an infection that is involving growth of bacteria or virus is proposed. This is particularly useful in individuals with diabetes mellitus or HIV either of which may increase the risk of infect. Infections from many microbial forms may raise IL-10 levels. Infection with Mycobacterium tuberculosis is an example of the ability of bacterial infection to raise Il-10 levels during infection. Other infections may include fungal infections such as Candida albicans. The ability of the oral ingestion of L-GSH to raise glutathione in the cells of individuals with HIV+ disease is an example of the ability oral L-GSH to support normal glutathione levels and maintain immune cell function in individuals with low glutathione. It is claimed that oral ingestion of L-GSH can return elevated levels of IL-10 to normal in individuals undergoing infection and allow for efficient management by the immune system of infection and chronic inflammation. This invention may be particularly useful in individuals with diabetes, or diabetes and tuberculosis, or in individuals with HIV, or with diabetes and HIV, or with diabetes, HIV and tuberculosis.

### CITATION LIST

1. Kwon DS, Kaufmann D E. Protective and detrimental roles of IL-10 in HIV pathogenesis. Eur Cytokine Netw. 2010;21(3):208-14. http://www.jle.com/en/revues/bio_rech/ecn/e-docs/000/04/5D/4A/article.phtml
2. Morris D, Guerra C, Khurasany M, Guilford F, Saviola B, Huang Y, et al. Glutathione supplementation improves macrophage functions in HIV. J Interferon Cytokine Res. 2013; 33(5):270-9. http://www.nchi.nlm.nih.gov/pubmed/23409922
3. Levitz S M, Tabuni A, Nong S H, Golenbock D T. Effects of interleukin-10 on human peripheral blood mononuclear cell responses to Cryptococcus neoformans, Candida albicans, and lipopolysaccharide. Infect Immun. 1996; 64(3):945-51. PMCID: 173861. http://www.ncbi.nlm.nih.gov/pubmed/8641805
4. School UoSCM. IMMUNOLOGY CHAPTER THIRTEEN—CYTOKINES AND IMMUNOREGULATION http://pathmicro.med.sc.edu/mobile/m.immuno-13.htm. MICROBIOLOGY AND IMMUNOLOGY MOBILE—University of South Carolina Medical School.
5. Mosser D M. The many faces of macrophage activation. J Leukoc Biol. 2003; 73(2):209-12.http://www.jleukbio.org/content/73/2/209.long
6. Romagnani S. T-cell subsets (Th1 versus Th2). Ann Allergy Asthma Immunol. 2000; 85(1):9-18; quiz , 21. http://www.ncbi.nlm.nih.gov/pubmed/10923599
7. Tubach F, Salmon D, Ravaud P, Allanore Y, Goupille P, Breban M, et al. Risk of tuberculosis is higher with anti-tumor necrosis factor monoclonal antibody therapy than with soluble tumor necrosis factor receptor therapy: The three-year prospective French Research Axed on Tolerance of Biotherapies registry. Arthritis Rheum. 2009;60(7):1884-94. PMCID: 2921546. http://www.ncbi.nlm/nih.gov/pubmed/19565495

1-14. (canceled)

15. A method of treatment of tuberculosis infected Type 2 diabetic patients having elevated IL-10 levels, comprising the following steps:
administering liposomally formulated reduced glutathione to Type 2 Diabetes mellitus patients with Tuberculosis infection having elevated IL-10 levels to modulate immune response and reduce IL-10 to normal levels of IL-10 for necessary levels of inflammatory response to preserve continued immune function.
